(19) United States
(12) Patent Application Publication
(10) Pub. No.: US 2014/0128767 A1
(43) Pub. Date: May 8, 2014

(54) EAR COUPLER

(71) Applicant: GN OTOMETRICS A/S, Taastrup (DK)

(72) Inventors: Morten HOEST, Ballerup (DK); Michael Frounberg JORGENSEN, Allerod (DK)

(73) Assignee: GN OTOMETRICS A/S, Taastrup (DK)

(21) Appl. No.: 14/151,614

(22) Filed: Jan. 9, 2014

Related U.S. Application Data
(63) Continuation-in-part of application No. 12/794,172, filed on Jun. 4, 2010.

(51) Int. Cl. A61B 5/12

(52) U.S. Cl. CPC A61B 5/125
USPC 600/559

(57) ABSTRACT

An ear coupler includes: a member having a cavity for accommodating at least a part of an ear; and a structure extending from the member, the structure having an end with a port; wherein the port is configured for detachably coupling to a transducer.

### RELATED APPLICATION DATA

This application is a continuation-in-part of U.S. patent application Ser. No. 12/794,172, filed on Jun. 4, 2010, pending, the entire disclosure of which is expressly incorporated by reference herein.

### FIELD

This invention relates to auditory devices, and more particularly, to audiometric devices and methods.

### BACKGROUND

A person may suffer from hearing impairment at an early age. Hearing impairment may occur in infants, and as early as when they are born. If hearing impairment is not detected early enough, then the child's language skills may be adversely affected, leading to potentially long-term disability. Thus, the most ideal time to test for an infant's hearing impairment is immediately after birth. Such early detection allows for early treatment. However, performing hearing tests on infants may be difficult because they cannot participate in conventional hearing tests, which require the subjects to provide feedback to indicate whether they can hear various stimulus signals.

Devices and methods have been used to evaluate infant hearing by subjecting the infant to an audio stimulus, and then measuring different responses, such as electroencephalographic or otoacoustic response, to the stimulus. Some existing hearing testing devices have a probe for insertion into an infant's ear. Such device would require a user to manually hold onto the probe to stabilize the device relative to the infant during use, which may be inconvenient for the user, especially if the performance of the hearing test require a lengthen period of time. Also, the probe of such device may not be comfortable for the infant's ear, since use of such device would require the probe to contact against the inner wall of the infant's ear canal. Furthermore, such device may not be safe for the infant since the distal tip of the probe may injure the ear canal of the infant.

### SUMMARY

An ear coupler includes: a member having a cavity for accommodating at least a part of an ear; and a structure extending from the member, the structure having an end with a port; wherein the port is configured for detachably coupling to a transducer.

A system includes the ear coupler; and the transducer.

Optionally, the transducer is configured to provide sound.

Optionally, the transducer is configured to obtain test data.

Optionally, the test data comprises otoacoustic emission test data.

Optionally, the system further includes: a testing device; wherein the transducer is configured for detachably coupling to the testing device.

Optionally, the testing device is configured to perform one or more of an otoacoustic emission test, an auditory brainstem response test, an acoustic reflectivity test, and a tympanometry test.

Optionally, the testing device is configured to recognize the transducer when the transducer is connected to the testing device.

Optionally, the testing device is configured to identify calibration data stored in the testing device based on the recognized transducer, and to apply the calibration data to audio data for transmission to the transducer.

Optionally, the testing device is configured to identify calibration data stored in the testing device based on the recognized transducer, and to apply the calibration data to test data received from the transducer.

Optionally, the testing device comprises an audiometric device.

Optionally, the system further includes a testing device configured to communicate with the transducer.

Optionally, the testing device comprises a communication component for wireless communication with the transducer.

Optionally, the testing device is configured to transmit audio data wirelessly to the transducer.

Optionally, the testing device is configured to receive test data wirelessly from the transducer.

Optionally, the transducer is a part of an assembly configured to communicate with a testing device, the assembly having a transmitter and/or a receiver.

Optionally, the transducer assembly further comprises a D-A converter functionally coupled to the transducer.

Optionally, the transducer is configured to receive test data, and the D-A converter is configured to convert the test data from analog to digital format before the transmitter transmits the test data to the testing device.

Optionally, the assembly further comprises a digital signal processor (DSP).

Optionally, the DSP is configured to process otoacoustic emission data.

Optionally, the receiver is configured to receive digital audio data from the testing device, and the DSP is configured to modify the audio data according to calibration data for the transducer.

Optionally, the member has a dome-shape.

Optionally, the member is transparent.

Optionally, a first part of the member is more transparent than a second part of the member.

Optionally, the structure comprises a tubular member.

Other and further aspects and features will be evident from reading the following detailed description of the embodiments, which are intended to illustrate, not limit, the invention.

### BRIEF DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIG. 1 illustrates a perspective view of an ear coupler in accordance with some embodiments.

FIG. 2 illustrates a cross sectional view of the ear coupler of FIG. 1.

FIG. 3 illustrates a plan view of the ear coupler of FIG. 1.

FIG. 4A illustrates a testing device with the ear coupler of FIG. 1.

FIG. 4B illustrates another testing device with the ear coupler of FIG. 1

FIG. 5 illustrates a variation of the ear coupler of FIG. 1 in accordance with other embodiments.

FIG. 6 illustrates a variation of the ear coupler of FIG. 1 in accordance with other embodiments.

FIG. 7 illustrates a variation of the ear coupler of FIG. 1 in accordance with other embodiments.

FIG. 8 illustrates a variation of the ear coupler of FIG. 1 in accordance with other embodiments.

FIG. 9 illustrates a variation of the ear coupler of FIG. 1 in accordance with other embodiments.

FIG. 10 illustrates an ear coupler system in accordance with some embodiments.

FIG. 11 illustrates an ear coupler system in accordance with other embodiments.

FIG. 12A illustrates another example of an ear coupler.

FIG. 12B illustrates another example of an ear coupler.

### DESCRIPTION OF THE EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

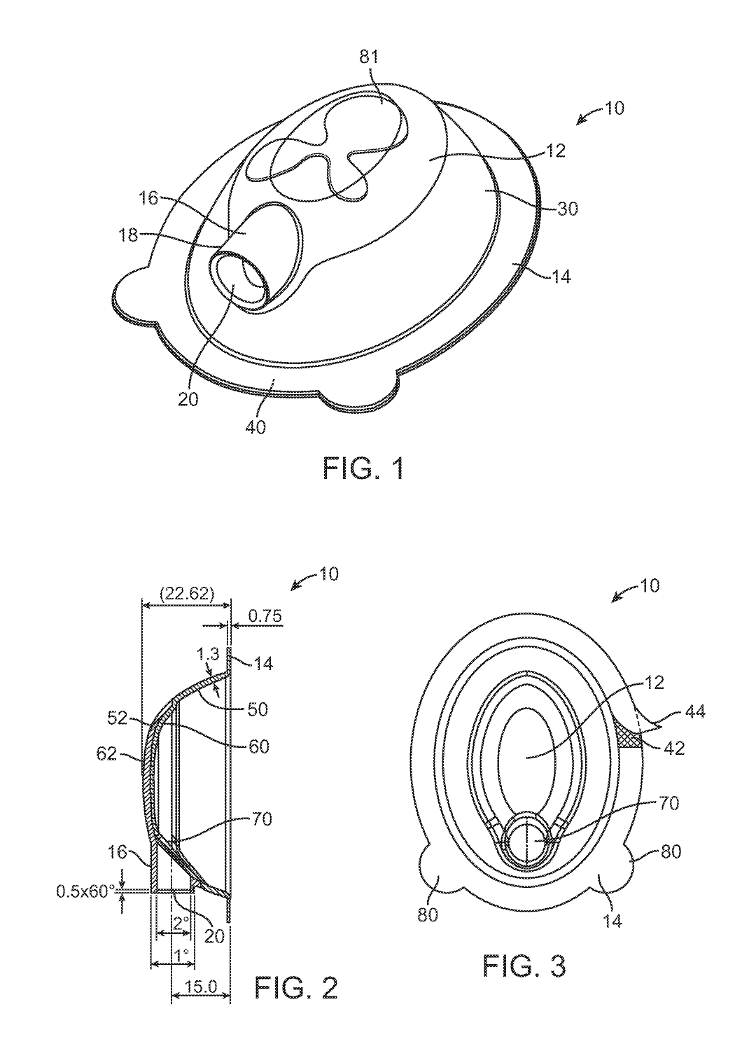

FIG. 1 illustrates an ear coupler 10 in accordance with some embodiments. The ear coupler 10 includes a dome-shape member 12, a flange 14, and a structure 16 having an end 18 with a port 20. The dome-shape member 12 includes a wall 30 from which the flange 14 extends. The flange 14 circumscribes around the member 12, and has a surface 40 with an adhesive 42 (shown in FIG. 3). The ear coupler 10 may further include a barrier 44 for containing the adhesive 42 before the ear coupler 10 is used (see FIG. 3, which illustrates part of the barrier 44 being removed from the flange 14).

FIG. 2 illustrates a cross section of the ear coupler 10 in accordance with some embodiments. As shown in the figure, the member 12 includes a first portion 50 and a second portion 52. The first portion 50 has a first wall portion 60, and the second portion 52 has a second wall portion 62 that overlaps the first wall portion 60 to thereby form at least a part of the wall 30 of the ear coupler 10. In the illustrated embodiments, the second portion 52 is made from a material that is stiffer than the first portion 50. For example, in some embodiments, the first portion 50 may be made from a material having a stiffness of 35 Shore A, and the second portion 52 may be made from a material having a stiffness of 45 Shore A. Such configuration is advantageous in that the second portion 52 may function as a stiffening element for strengthening at least a part of the member 12. In other embodiments, the first and second portions 50, 52 may be made from the same material. Each of the first and second portions 50, 52 may be made from a variety of materials, including but not limited to TPE (Thermoplastic elastomers), Silicone rubber, or any of other types of rubber or polymers. Also, in further embodiments, the second portion 52 may be larger so that it completely overlaps the entire first portion 51.

The second portion 52 may be overmolded with the first portion 50 during a manufacturing process. In such cases, the first portion 50 and the second portion 52 are fused together. In other embodiments, the first and second portions 50, 52 may be attached together by other techniques, such as via an adhesive, a screw, a snap-fit coupler, or other types of mechanical connections.

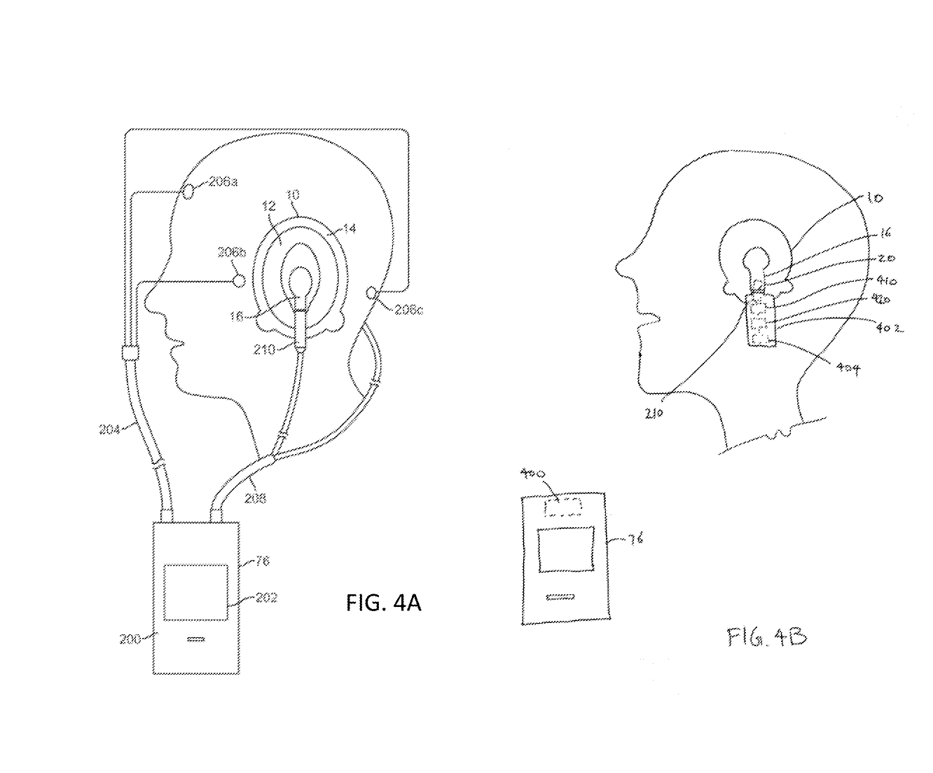

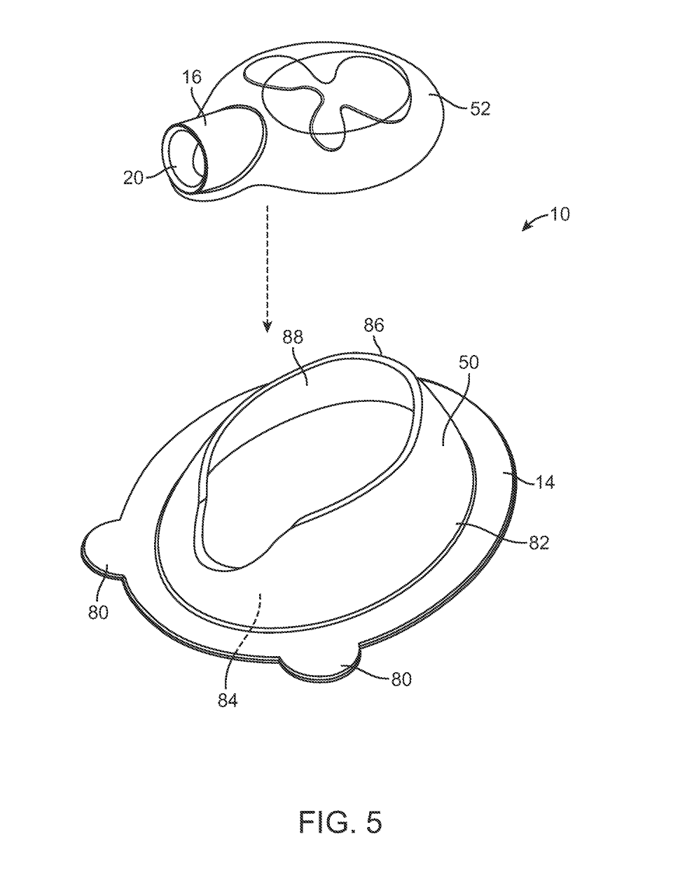

As shown in the illustrated embodiments, the wall 30 of the member 12 has an opening 70, which corresponds with the structure 16. The structure 16 has a tubular configuration to thereby define the port 20 at the end 18 of the structure 16. In the illustrated embodiments, the port 16 has a circular cross section. In other embodiments, the port 16 may have other cross sectional shapes, such as an elliptical shape, a square shape, a rectangular shape, or other shapes, such as a customized shape. The port 16 is sized and shaped so that it allows an audio device 76 (shown in FIG. 4A), such as an audiometric device, to couple thereto.

FIG. 3 illustrates a plan view of the ear coupler 10. As shown in the figure, the ear coupler 10 also includes a plurality of tabs 80 extending from the flange 14. The tabs 80 allow a user to decouple the ear coupler 10 from a subject by holding onto one of the tabs 80, and pulling the flange 14 away from the subject's skin. In the illustrated embodiments, the ear coupler 10 has two tabs 80. In other embodiments, the ear coupler 10 may have more than two tabs 80, or less than two tabs 80 (i.e., one tab 80). In further embodiments, the tab(s) 80 are optional, and the ear coupler 10 does not include any tab 80. In such cases, the ear coupler 10 may be decoupled from the subject by pulling the flange 14 away from the subject's skin.

In some embodiments, the entire ear coupler 10 is transparent. Alternatively, part of the ear coupler 10 may have one or more color. For example, part of the ear coupler 10 may include a graphic 81 that is in color. Also, in other embodiments, a part of the ear coupler 10 may be more transparent than another part of the ear coupler 10. For example, in other embodiments, the first portion 50 and the second portion 52 may be both transparent, with the first portion 50 being more transparent than the second portion 52. The transparency allows a user to see at least a part of a subject's ear while the user is putting the ear coupler 10 onto the subject, so that the user can place the ear coupler 10 at a desired position relative to the subject's ear.

As discussed, the ear coupler 10 is configured for use with an audio device, such as an audiometric device. In some embodiments, the audiometric device may be configured to perform one or more tests to test a hearing of a subject, such as an infant. Examples of tests that may be performed using the audiometric device include an otoacoustic emission test, an auditory brainstem response test, an acoustic reflectivity test, and a tympanometry test. As shown in FIG. 4A, in one implementation, the audiometric device 76 includes a handheld unit 200 with a display screen 202, a first cable 204, and a second cable 208. The first cable 204 has three electrodes 206a-206c that are configured to be coupled to the subject's forehead, cheekbone, and nape of the neck, respectively, during use. In other embodiments, any of the electrodes 206 may be placed at a location that is different from the examples described. For example, in other embodiments, any of the electrodes 206 may be placed at a temple, a shoulder, etc. The second cable 208 has two transducers 210 (one is shown) for detachably coupling to two respective ear couplers 10 (one is shown) during use. The audiometric device 76 is configured to generate one or more stimulus signals, which are transmitted to the subject's ears via the ear couplers 10. The stimulus signal(s) is for evoking a neural response, which is measured by the electrodes 206a-206c, to thereby perform the auditory brainstem response test. The same stimulus signal(s), or other stimulus signal(s), may be used for evoking another response for the otoacoustic emission test, which is measured by the transducer 210. The display 202 is configured to display information relating to the hearing tests, such as parameters for conducting the tests, and test results.

FIG. 4A illustrates a method of using the ear coupler 10 in accordance with some embodiments. During use, the barrier 44 is removed to expose the adhesive 42 on the flange 14. The ear coupler 10 is then attached to a subject's skin via the adhesive 42. If part of the ear coupler 10 is transparent, then the user of the ear coupler 10 can view through the ear coupler 10 to see the subject's ear, so that the user can center the ear coupler 10 with respect to the subject's ear. The audio device 76 is detachably coupled to the port 20 of the ear coupler 10 during use. In particular, the transducer 210 of the audio device 76 is detachably coupled to the ear coupler 10 via the port 20. Another transducer (not shown) that connects to the audio device 76 is also coupled to another ear coupler (not shown). Also, the electrodes 206a-206c from the audio device 76 are secured to the subject. In the illustrated embodiments, the audio device 76 is an audiometric device configured to perform an otoacoustic emission test and an auditory brainstem response test on the subject. In some embodiments, the audio device 76 is configured to provide a stimulus signal via the cable 208 to evoke a first response and a second response from the subject, wherein the first response (measured using the transducer 210) is for the otoacoustic emission test, and the second response (measured using the electrodes 206a-206c) is for the auditory brainstem response test. In other embodiments, the audio device 76 is configured to provide a first stimulus signal to evoke a first response for the otoacoustic emission test, and a second stimulus signal to evoke a second response for the auditory brainstem response test.

Also, instead of using ear couplers 10 to perform both the otoacoustic emission test and the auditory brainstem response test, the ear couplers 10 may be used to perform only one of the otoacoustic emission test and the auditory brainstem response test. In such cases, when performing the other one of the otoacoustic emission test and the auditory brainstem response test, the ear couplers 10 may be removed from the subject and detached from the audio device 76 (e.g., by detaching the ear couplers 10 from the transducers 210, or by detaching the cable 208 from the audio device 76). A probe (not shown) can then be attached to the audio device 76 (e.g., by attaching the probe to the transducer 210), and the probe is inserted into a subject's ear for delivering stimulus signal(s) for performing the other test.

As illustrated, the ear coupler 10 is advantageous because it obviates the need to insert a probe into a subject's ear during a hearing test, which may be uncomfortable for the subject, and/or may injure the subject's ear. Also, because the ear coupler 10 is capable of securing itself to the subject, use of the ear coupler 10 does not require the user to manually stabilize the ear coupler 10 relative to the subject's ear. Furthermore, the dome shape of the member 12 renders the member 12 less susceptible (e.g., compared to a member 12 with a flat cover) to deformation and damage because of the arc-action provided by the dome shape in response to externally applied pressure or force. In addition, providing the port 20 via the structure 16 is more advantageous than providing a coupling port at the wall 30 of the member 12 for coupling to the audio device 76. This is because the structure 16 functions as a stiffening device that enhances the integrity of the port 20. If the device 10 does not include the structure 16, the transducer 210 will need to be directly coupled to the ear coupler 10 via a port at the wall 30, which weakens the wall 30. Such configuration will also subject the wall 30 to bending during insertion and removal of the transducer 210 relative to the wall 30, which is undesirable. In some embodiments, the opening 70 is configured to be larger than the port 20 so that when the transducer 210 is inserted into the port 20, the transducer 210 will not touch the wall portion that defines the opening 70. This has the benefit of ensuring that the structural integrity of the wall portion that defines the opening 70 is preserved.

As discussed above, the transducer 210 may be configured to obtain test measurement data (e.g., response from a user, such as sound reflected from a user, sound reflected from a user's tympanic membrane, etc.) and/or to provide audio data (e.g., stimulus signals) for output into the user's ear. In some embodiments, the audio device (testing device) 76 may be configured to recognize the transducer 210 when the transducer 210 is connected to the testing device 76. For example, the transducer 210 may be detachably coupled to the distal end of the cable 208, and the testing device 76 may automatically recognize the transducer 210 when the transducer 210 is coupled to the distal end of the cable 208. In another example, the proximal end of the cable 208 may be detachably coupled to the testing device 76. In such cases, the transducer 210 may be coupled (detachably or permanently coupled) to the distal end of the cable 208. When the proximal end of the cable 208 is detachably coupled to the testing device 76, the testing device 76 may automatically recognize the transducer 210 that is attached to the distal end of the cable 208.

Furthermore, in some embodiments, after the testing device 76 has recognized the transducer 210, the testing device 76 may retrieve calibration data stored in the testing device 76 based on the recognized transducer 210. The testing device 76 may then apply the calibration data to audio data (e.g., stimulus signal) before the audio data is transmitted from the testing device 76 to the transducer 210 for output to the user's ear. In one implementation, the testing device 76 may have a non-transitory medium storing different calibration data for different transducers 210 or different types of transducers 210. In such cases, when a certain transducer 210 is detachably coupled to the testing device 76, the testing device 76 then recognizes the transducer 210 and retrieves the corresponding calibration data from the non-transitory medium.

In some embodiments, the calibration data may be level(s) or gain(s), frequency or frequencies, time constant(s), information regarding any of the foregoing, and/or any combination of the foregoing. Any of the foregoing information, or any combination of the foregoing information, may be used by the testing device 76 to control an output of the transducer. For example, in one implementation, the calibration data may be applied to audio data before the audio data is output from the transducer to thereby control an output of the transducer. In some embodiments, the calibration data allow different types of transducer to provide substantially the same output (e.g., outputs that differ in at least one respect by less than 10%). In other embodiments, the calibration data allow different types of ear coupler to achieve substantially the same sound-coupling effect (e.g., effects that differ in at least one respect by less than 10%). In further embodiments, the calibration data allow different “transducer-ear coupler” combinations to provide substantially the same output (e.g., outputs that differ in at least one respect by less than 10%). Also, in some embodiments, the calibration data may be configured (e.g., by having certain values) to compensate for differences in the cavity size and/or cavity shape between the different ear couplers, and/or to compensate for the different coupling mechanisms (transfer functions) between the different ear couplers and ear, so that the different ear couplers achieve substantially the same sound-coupling effect (e.g., effects that differ in at least one respect by less than 10%). In addition, in some embodiments, the calibration data may be configured based on certain feature(s) of a transducer, and/or certain features (e.g., cavity size, and/or cavity shape, etc.) of an ear coupler.

In other embodiments, the calibration data may be applied to test data received from the transducer.

In the above embodiments, the testing device 76 is configured to communicate with the transducer 210 via the cable 208. In other embodiments, the testing device 76 may be configured to communicate wirelessly with the transducer 210. FIG. 4B illustrates a variation of the system of FIG. 4A. In particular, the testing device 76 of FIG. 4B is the same as that of FIG. 4A, except that the testing device 76 of FIG. 4B includes a wireless device 400 (instead of the cable 208) for communication with the transducer 210. The transducer 210 is a part of an assembly 402 that includes a wireless device 404 for communication with the wireless device 400 of the testing device 76. The wireless device 404 at the assembly 402 may be a receiver, a transmitter, or a combination of both (e.g., a transceiver). In other embodiments, the wireless device 404 may be located outside the assembly 402. For example, the wireless device 404 may be located anywhere along the cable 208, or at a connecting portion between the cable 208 and the assembly 402. In the illustrated embodiments, the assembly 402 also includes a D-A converter 410.

In some embodiments, when the assembly 402 is detachably coupled to the port 20 at the structure 16 of the ear coupler 10, the transducer 210 (or at least a part of it) may be located in the structure 16 like that shown in the example. In other embodiments, when the assembly 402 is detachably coupled to the port 20, the transducer 210 may be located outside the structure 16. For example, the transducer 210 may be accommodated completely inside the assembly 402.

In some embodiments, the testing device 76 is configured to transmit audio data (e.g., stimulus signal) generated at the testing device 76 wirelessly to the assembly 402 using the wireless device 400 at the testing device 76. The transducer 210 receives the audio data using the wireless device 404 at the assembly 402, and provides stimulus signals to the user based on the audio data. In one implementation, the transducer 210 may be a part of a speaker that outputs sound to the user.

Also, in some embodiments, the transducer 210 may receive test data (e.g., sound returned) from the user. After the transducer 210 receives the test data, the D-A converter 410 converts the test data from analog to digital format before the transmitter 404 at the assembly 402 transmits the test data to the testing device 76.

For example, in some embodiments, the transducer 210 may be configured to pick up sound returned from an inner ear (e.g., like an echo) of the user. The returned sound may be used to obtain Otoacoustic Emission (OAE) measurements. In some embodiments, the transducer 210 may be located outside the ear canal and at the entrance of the ear canal. In other embodiments, the transducer 210 may be located in the ear canal. In such cases, the transducer 210 may be at an end of a probe that is a part of the assembly 402. During use, the probe with the transducer 210 may be inserted through an opening of the ear coupler 10 until a connector at the assembly 402 reaches a connecting portion (e.g., end 18 of structure 16) of the ear coupler 10 and is connected to the ear coupler 10. When the ear coupler 10 is placed over the user's ear, the probe with the transducer 210 is then accordingly be placed in the ear canal. In further embodiments, the transducer 210 may be located at the outer ear.

In other embodiments, instead of having the transducer 210 both providing audio data and receiving test data, the assembly 402 may have separate components for performing the different respective functions. For example, the assembly 402 may have a speaker for providing stimulus signals, and a transducer for receiving test data from a user.

In the illustrated embodiments, the assembly 402 also includes a digital signal processor (DSP) 420. In some embodiments, the DSP 420 may be configured to process the test data obtained from the user. In particular, after the transducer 210 receives test data (e.g., returned sound) from the user, the D-A converter 410 converts the test data from analog format into digital format. The D-A converter 410 then passes the digitized test data to the DSP 420 for processing. In some embodiments, the test data may be otoacoustic emission data, and the DSP 420 may be configured to process the otoacoustic emission data. In other embodiments, the test data may be other types of test data obtained from the user. Also, in some embodiments, processed test data may be transmitted from the assembly 402 to the testing device 76 using the wireless components 400, 404. In other embodiments, processed test data may be stored in a non-transitory medium inside the assembly 402.

In other embodiments, instead of, or in addition to, the DSP 420 being configured to process the test data obtained from the user, the DSP 420 may be configured to receive digital audio data from the testing device 76, and process the digital audio data before passing it to the transducer 210 for presentation to the user. For example, in some embodiments, the DSP 420 may be configured to modify the audio data according to calibration data for the transducer 210.

Also, in other embodiments, instead of being at the assembly 402, the DSP 420 may be at the testing device 76.

It should be noted that the testing device 76 may be any audio device, such as an audiometric device, or any of other types of device that can perform a test on a user.

In the above embodiments, the first portion 50 has a dome shape, and the second portion 52 overlaps part of the wall of the first portion 50. In other embodiments, the first portion 50 may have different configurations. For example, as shown in FIG. 5, in other embodiments, the first portion 50 may have a first end 82 with a first opening 84, and a second end 86 with a second opening 88. The first end 82 is attached to (e.g., integral with) the flange 14.

The second portion 52 (which includes the structure 16) is configured to cover the opening 88 at the second end 86 of the first portion 60, thereby forming the dome shape member 12. In some embodiments, the second portion 52 may be overmolded onto the first portion 50. Alternatively, the second portion 52 may be attached to the first portion 50 by an adhesive, a screw, a snap-fit connector, or other types of connectors.

In further embodiments, the second portion 52 may be configured to be detachably coupled to the first portion 50 (e.g., via a clip, threads, a snap-fit connector, friction, etc.), thereby allowing the second portion 52 to function like a removeable cover. Such configuration is advantageous in that it allows a user to selectively open the cover 52 to directly view the subject's ear, and/or to directly communicate to the subject. Such configuration also allows a user to remove the cover 52 with the structure 16, and attach another cover 52 to the rest of the ear coupler 10. This is advantageous because it would allow the user to replace the cover 52 if it is broken. In such cases, the ear coupler 10 includes a plurality of covers 52 with a same size and shape. In other embodiments, the detachable configuration also allows a user to replace the cover 52 with a different configuration of the structure 16 (e.g., a cover 52 with a structure 16 having a different size and/or shape for coupling to a different audio device). In such cases, the ear coupler 10 may include a plurality of covers 52 with respective structures 16 having different configurations (e.g., sizes and/or shapes), wherein each structure 16 is configured to detachably couple to the rest of the ear coupler 10 at one end, and to an audio device at the other end of the structure 16. It should be noted that the cover 52 may be considered to be a part of the ear coupler 10, or a separate component that is configured to be coupled to the ear coupler 10.

In the illustrated embodiments, the first and second portions 50, 52 may be made from the same material. Alternatively, the second portion 52 may be made from a material that is different from that of the first portion 50. For example, in some embodiments, the second portion 52 may be made from a material that is stiffer than that of the first portion 50.

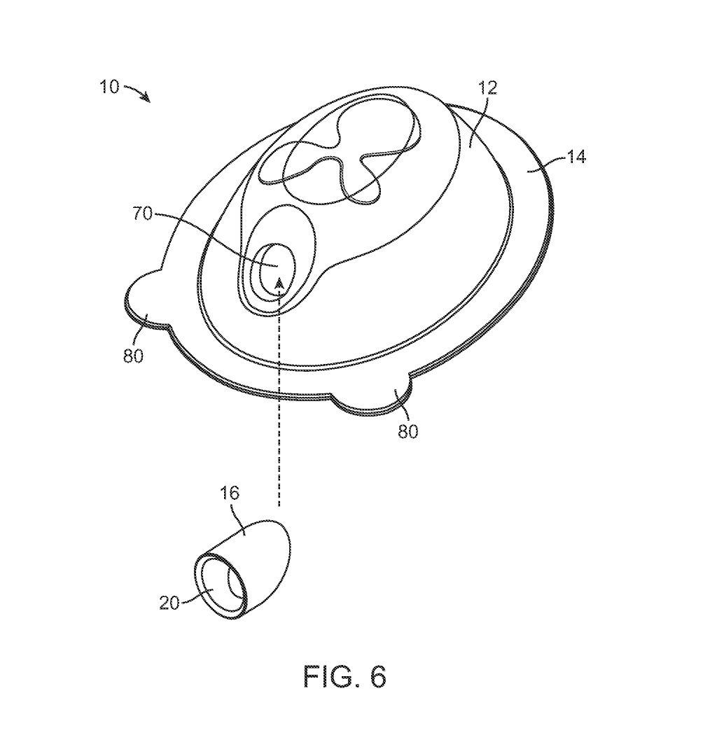

In other embodiments, the member 12 and the flange 14 of the ear coupler 10 may be formed as one piece during a manufacturing process, and the structure 16 is a separate component that is attached to the rest of the ear coupler 10 (FIG. 6). For example, the structure 16 may be overmolded onto the wall 30 of the member 12. Alternatively, the structure 16 may be attached to the member 12 by an adhesive, a screw, a snap-fit connector, or other types of connectors. In the illustrated embodiments, the member 12 and the structure 16 may be made from the same material. Alternatively, the structure 16 may be made from a material that is different from that of the member 12. For example, in some embodiments, the structure 16 may be made from a material that is softer than that of the member 12.

In further embodiments, the structure 16 may be configured to be detachably coupled to the member 12 (e.g., via a clip, threads, a snap-fit connector, friction, etc.). Such configuration allows a user to remove the structure 16 from the member 12, and attach another structure 16 to the rest of the ear coupler 10. This is advantageous because it would allow the user to replace the structure 16 if it is broken. In such cases, the ear coupler 10 may include a plurality of structures 16 that have a same size and shape. In other embodiments, the detachable configuration also allows a user to replace the structure 16 with another structure 16 with a different configuration (e.g., a structure 16 with a different size and/or shape for coupling to a different audio device). In such cases, the ear coupler 10 may include a plurality of structures 16 with different configurations (e.g., sizes and/or shapes), wherein each structure 16 is configured to detachably couple to the rest of the ear coupler 10 at one end, and to an audio device at the other end of the structure 16.

In some embodiments, the structure 16 may be made from a bendable material that can be bent by a user during use. Such configuration allows the user to selectively position the audio device (that is coupled to the ear coupler 10) so that the audio device is at a desired position relative to the ear coupler 10.

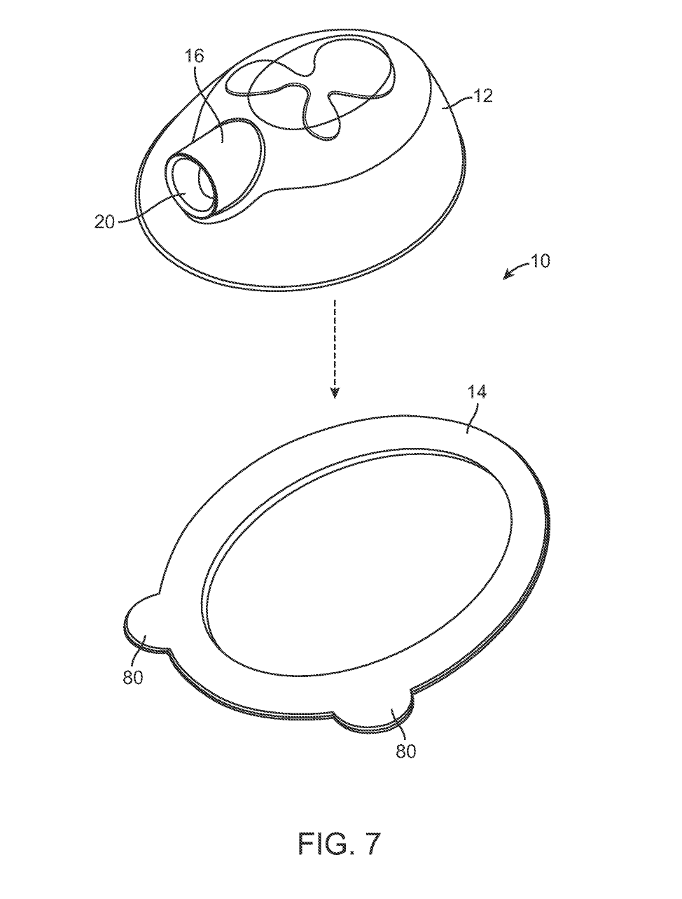

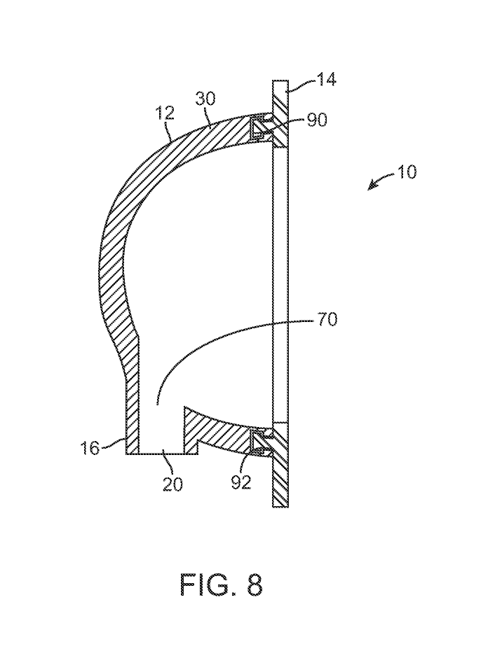

In other embodiments, the wall 30 and the structure 16 of the ear coupler 10 may be formed as one piece during a manufacturing process, and the flange 14 is a separate component that is attached to the rest of the ear coupler 10 (FIG. 7). For example, the flange 14 may be overmolded onto the wall 30 of the member 12. Alternatively, the flange 14 may be attached to the member 12 by an adhesive, a screw, a snap-fit connector, or other types of connectors. In further embodiments, the wall 30 of the member 12 may include a slot 90, and the flange 14 may include a protrusion 92 configured to mate with the slot 90 (FIG. 8), thereby securing the flange 14 relative to the member 12. In other embodiments, the slot-protrusion configuration may be reversed—i.e., the flange 14 may have a slot, and the wall 30 of the member 12 may have a protrusion for mating with the slot at the flange 14.

In further embodiments, the flange 14 may be configured to be detachably coupled to the member 12 (e.g., via a clip, threads, a snap-fit connector, friction, etc.). Such configuration allows a user to remove the flange 14 from the member 12, and attach another flange 14 to the rest of the ear coupler 10. In some embodiments, the ear coupler 10 may include a plurality of flanges 14 that have a same size and shape. This is advantageous because it would allow the user to replace the flange 14 if it is broken, or when the adhesive 42 becomes non-sticky. Alternatively, or additionally, the ear coupler 10 may include a plurality of flanges 14 that have different configurations (e.g., sizes and/or shapes), wherein each flange 14 is configured to detachably couple to the member 12 of the ear coupler 10. Such system allows a user to replace the flange 14 with another flange 14 with a different configuration (e.g., a flange 14 with a different size and/or shape).

In the illustrated embodiments, the member 12 and the flange 14 may be made from the same material. Alternatively, the flange 14 may be made from a material that is different from that of the member 12. For example, in some embodiments, the flange 14 may be made from a material that is softer than that of the member 12. Such configuration allows the flange 14 to conform to a profile of the subject's skin during use. In some embodiments, the flange 14 may be made from a compliant material that can be easily bent or deform upon pressing the flange 14 against the subject's skin.

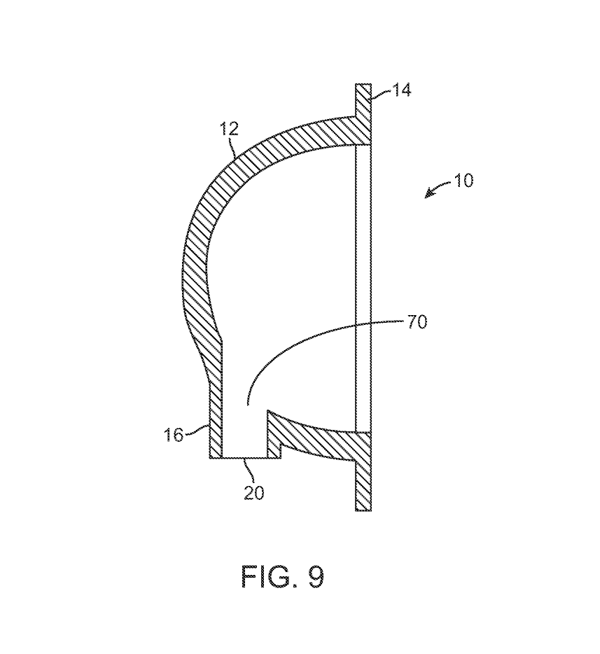

In further embodiments, the ear coupler 10 may be made from one material, and the entire ear coupler 10 may be integrally formed as one piece to have an unity configuration. FIG. 9 illustrates another ear coupler 10 in accordance with other embodiments. As shown in the figure, the entire ear coupler 10, including the wall 30 of the member 12 of the ear coupler 10, the flange 14, and the structure 16, is formed (e.g., molded) as one piece during a manufacturing process.

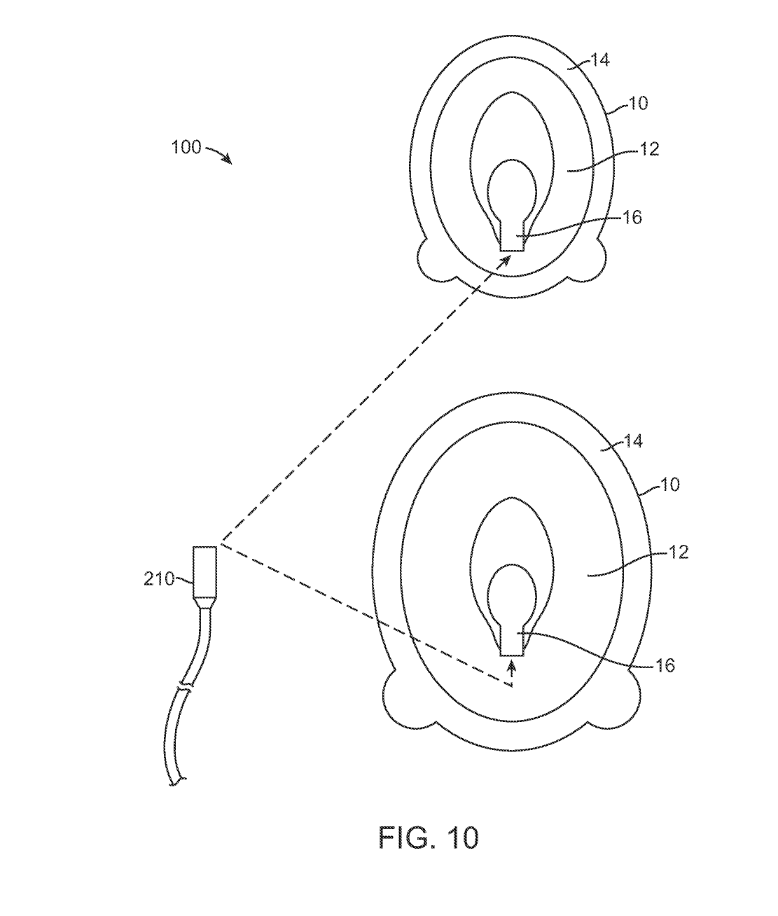

FIG. 10 illustrates an ear coupler system 100 that includes a plurality of ear couplers 10 with different configurations. Each of the ear couplers 10 may have any of the configurations described herein. In the illustrated embodiments, the ear couplers 10 have different sizes for accommodating different subjects' ears with different respective sizes. In other embodiments, the ear couplers 10 have different shapes for accommodating different subjects' ears with different respective shapes. Each of the ear couplers 10 has a structure 16 with a port 20 for detachably coupling to a connector 210 of an audio device, such as the audiometric device 76 of FIG. 4A or 4B. In the figure, one ear coupler of each size is shown. However, it should be understood that in some embodiments, the system 100 may provide pairs (one for left ear and one for right ear) of ear couplers 10 in different sizes (e.g., a pair of ear couplers 10 in one size, and another pair of ear couplers 10 in another size). Also, in further embodiments, the system 100 may include more that two pairs of ear couplers 10.

In other embodiments, as shown in FIG. 11, each of the ear couplers 10 may have the configuration shown in FIG. 5. In such cases, the same cover 52 may be used to detachably couple the audio device to a selected one of the ear couplers 10. In the figure, one ear coupler of each size is shown. However, it should be understood that in some embodiments, the system 100 may provide pairs (one for left ear and one for right ear) of ear couplers 10 in different sizes (e.g., a pair of ear couplers 10 in one size, and another pair of ear couplers 10 in another size). Also, in further embodiments, the system 100 may include more that two pairs of ear couplers 10.

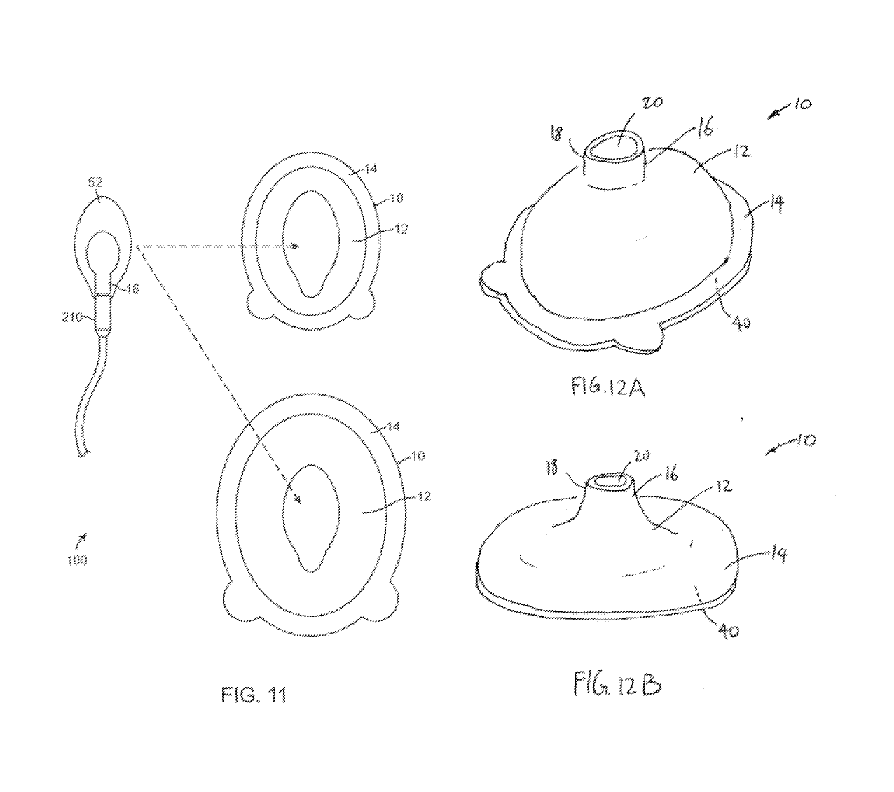

It should be noted that the ear coupler 10 is not limited to the examples described previously, and that the ear coupler 10 may have other configurations in other embodiments. FIG. 12A illustrates another example of an ear coupler 10. The ear coupler 10 is similar to the embodiment of FIG. 1, except that the structure 16 with the port 20 at the end 18 of the structure 16 is oriented differently. In the illustrated embodiments, the structure 16 is a tubular member that extends from a top of the ear coupler 10. The port 20 is configured (e.g., sized and/or shaped) to detachably coupled to a device, like the transducer 210 or the assembly 402 described with reference to FIGS. 4A and 4B. In some embodiments, the assembly 402 may have a portion configured for insertion into the port 20 of the structure 16 at the ear coupler 10. In other embodiments, the assembly 402 may have a portion with an opening configured for allowing the end of the structure with the port 20 to insert therein.

FIG. 12B illustrates another example of an ear coupler 10. The ear coupler 10 is similar to the embodiment of FIG. 12A, except that the dome-shape member 12 has a different configuration. In the illustrated embodiments, the side profile of the member 12 extends from the top to the bottom in an exponential configuration, such that a slope of the side profile is steeper at the top in comparison with the slope of the side profile at the bottom. Also, instead of forming an angle between the member 12 and the flange 14, the member 12 transitions smoothly into the flange 14. The flange 14 has a bottom surface 40 with an adhesive for detachably coupling to a skin of a user. In some embodiments, the member 12 may be sized to accommodate an entire ear of the user. In other embodiments, the member 12 may be sized to accommodate a part of the ear of the user. In such cases, the flange 14 may cover a remaining part of the ear of the user. As similarly discussed with the previous embodiments, the port 20 is configured (e.g., sized and/or shaped) to detachably coupled to a device, like the transducer 210 or the assembly 402 described with reference to FIGS. 4A and 4B. In some embodiments, the assembly 402 may have a portion configured for insertion into the port 20 of the structure 16 at the ear coupler 10. In other embodiments, the assembly 402 may have a portion with an opening configured for allowing the end of the structure with the port 20 to insert therein.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. The specification and drawings are, accordingly, to be regarded in an illustrative rather than restrictive sense. The present inventions are intended to cover alternatives, modifications, and equivalents, which may be included within the spirit and scope of the present inventions as defined by the claims.

What is claimed:

1. An ear coupler, comprising:
a member having a cavity for accommodating at least a part of an ear; and
a structure extending from the member, the structure having an end with a port;
wherein the port is configured for detachably coupling to a transducer.

2. A system comprising the ear coupler of claim 1; and
the transducer.

3. The system of claim 2, wherein the transducer is configured to provide sound.

4. The system of claim 2, wherein the transducer is configured to obtain test data.

5. The system of claim 4, wherein the test data comprises otoacoustic emission test data.

6. The system of claim 2, further comprising:
a testing device;
wherein the transducer is configured for detachably coupling to the testing device.

7. The system of claim 6, wherein the testing device is configured to perform one or more of an otoacoustic emission test, an auditory brainstem response test, an acoustic reflectivity test, and a tympanometry test.

8. The system of claim 6, wherein the testing device is configured to recognize the transducer when the transducer is connected to the testing device.

9. The system of claim 8, wherein the testing device is configured to identify calibration data stored in the testing device based on the recognized transducer, and to apply the calibration data to audio data for transmission to the transducer.

10. The system of claim 8, wherein the testing device is configured to identify calibration data stored in the testing device based on the recognized transducer, and to apply the calibration data to test data received from the transducer.

11. The system of claim 6, wherein the testing device comprises an audiometric device.

12. The system of claim 2, further comprising a testing device configured to communicate with the transducer.

13. The system of claim 12, wherein the testing device comprises a communication component for wireless communication with the transducer.

14. The system of claim 13, wherein the testing device is configured to transmit audio data wirelessly to the transducer.

15. The system of claim 13, wherein the testing device is configured to receive test data wirelessly from the transducer.

16. The system of claim 2, wherein the transducer is a part of an assembly configured to communicate with a testing device, the assembly having a transmitter and/or a receiver.

17. The system of claim 16, wherein the transducer assembly further comprises a D-A converter functionally coupled to the transducer.

18. The system of claim 17, wherein the transducer is configured to receive test data, and the D-A converter is configured to convert the test data from analog to digital format before the transmitter transmits the test data to the testing device.

19. The system of claim 17, wherein the assembly further comprises a digital signal processor (DSP).

20. The system of claim 19, wherein the DSP is configured to process otoacoustic emission data.

21. The system of claim 19, wherein the receiver is configured to receive digital audio data from the testing device, and the DSP is configured to modify the audio data according to calibration data for the transducer.

22. The ear coupler of claim 1, wherein the member has a dome-shape.

23. The ear coupler of claim 1, wherein the member is transparent.

24. The ear coupler of claim 1, wherein a first part of the member is more transparent than a second part of the member.

25. The ear coupler of claim 1, wherein the structure comprises a tubular member.
